(19) United States
(12) Patent Application Publication
(10) Pub. No.: US 2025/0276136 A1
(43) Pub. Date: Sep. 4, 2025

(54) DISPENSER AND EVALUATION UNIT THEREFOR

(71) Applicants: Aptar Radolfzell GmbH, Radolfzell (DE); APTAR RADOLFZELL GMBH, Radolfzell (DE)

(72) Inventors: Michael HELMLINGER, Radolfzell-Böhringen (DE); Jörg KOHNLE, Villingen-Schwenningen (DE)

(21) Appl. No.: 18/859,758

(22) PCT Filed: Apr. 6, 2023
(86) PCT No.: PCT/EP2023/059212

§ 371 Date: Oct. 24, 2024

(30) Foreign Application Priority Data
Apr. 26, 2022 (EP) 22170084.2

(51) Int. Cl. A61M 11/00

(52) U.S. Cl. CPC A61M 11/008; A61M 11/007; A61M 2202/064; A61M 2205/3592

(57) ABSTRACT

A dispenser for pharmaceutical or cosmetic media including a housing, a media reservoir and a discharge opening for delivering medium from the media reservoir to the surrounding atmosphere. The dispenser further includes an electronic evaluation unit for detecting use of the dispenser, and the electronic evaluation unit is configured for detachable attachment to the dispenser housing and has a contact side. A magnetic or magnetizable holding surface is provided on an outer side of the dispenser housing, and the contact side of the evaluation unit is magnetic or magnetizable such that the evaluation unit is magnetically fastenable on the housing.

### FIELD OF APPLICATION AND PRIOR ART

The invention relates to a dispenser for discharging pharmaceutical or cosmetic media and in particular to an electronic evaluation unit for such a dispenser.

A dispenser of the generic type serves for discharging media, in particular of pharmaceutical liquids. It is known to provide such dispensers with evaluation units which detect the use of the dispenser via a sensor system, for example in order to permit a subsequent check as to whether a medicament is taken correctly and in accordance with a medication plan.

Known evaluation units are structurally integrated in the respective dispenser or are detachably attached to the dispenser, so that they can be detached from the dispenser and attached to another dispenser of the same type.

In the case of detachable evaluation units, a coupling mechanism is required in order to attach it to the housing of the dispenser.

### Problem and Solution

The invention addresses the problem of providing an evaluation unit for a dispenser which permits convenient handling in the course of disassembly and assembly on a dispenser.

According to the invention, a dispenser for discharging pharmaceutical or cosmetic media which has an electronic evaluation unit is proposed. The invention also relates furthermore to the evaluation unit as such.

A dispenser according to the invention has a housing which forms the outer contour of the dispenser and can be of a multi-part design. In particular, such a housing may have a reservoir body which forms the media reservoir and in particular a liquid reservoir in which the medium, in particular a pharmaceutical liquid, is stored before discharge.

The dispenser has a discharge opening through which medium from the media reservoir can be delivered into a surrounding atmosphere.

The dispenser is preferably designed for dispensing liquid, in particular in the form of a jet, a spray jet, or in the form of individual droplets. Instead, the dispenser may however also be intended for other media, for example in particular for dispensing powder for the purpose of inhalation.

If the dispenser is designed as a liquid dispenser, it may in particular be a pump dispenser which has a manually actuable pumping device which conveys liquid from the liquid reservoir to the discharge opening. Alternatively, it is a squeeze-bottle dispenser which has an elastically deformable reservoir body, by the manual compression of which liquid can be discharged.

The dispenser may furthermore be designed as an inhaler, in particular as a dispenser with a container which can be depressed for delivering a dose of the liquid, for the delivery of a defined dose of the medicament. According to a further example, the dispenser may be designed as a nasal dispenser, in particular with an elongate nasal applicator, at the distal end of which the discharge opening is provided.

The electronic evaluation unit is designed for detecting the use of the dispenser. For this, it has electrical or electronic components, in particular a battery, a processor and at least one sensor. The sensor is used to capture data which allow conclusions to be drawn about the use of the dispenser. In this case, the sensors can be designed to directly detect an external application of force, such as occurs for example when a hand-operated actuator for carrying out a pumping actuation is depressed. Preferably, however, at least one sensor which can detect data without contact and without external application of force is provided, it being possible for it to be in particular a microphone and/or a movement sensor. For example, the output data of a microphone may be evaluated in order to detect characteristic noises during use.

The evaluation unit preferably comprises an output device, in particular in the form of at least one LED, a display, a vibrator and/or a loudspeaker, in order to output signals to the user, for example in order to transmit the information that a discharge has been sensed or is pending according to the medication plan.

The evaluation unit is preferably designed to be used together with an external device such as in particular a smartphone. For communication with the external device, the evaluation unit preferably has a radio module, in particular a Bluetooth and/or WIFI radio module or else a GSM/3G/4G or 5G radio model.

In order to be able to quickly and conveniently separate the evaluation unit from the housing of the dispenser and couple it to a new dispenser, according to a first aspect of the invention it is proposed that a magnetic or magnetizable holding surface is provided on the outer side of the housing of the dispenser, in particular on a reservoir body of the housing. A contact side of the evaluation unit is correspondingly designed as magnetic or magnetizable such that the evaluation unit can be magnetically fastened on the housing.

The magnetic connection provided according to the invention makes the attachment of the evaluation unit possible very easily and conveniently. As soon as the evaluation unit is brought into the region of the housing that is provided for the attachment of the evaluation unit, the evaluation unit is magnetically attracted and remains fixed to the housing of the dispenser in the intended position.

In addition to the convenience of handling, with a suitable design and in particular size of the holding surface on the housing, it is also achieved in this way that the evaluation unit assumes a predefined position quite closely. This represents an advantage, since this predefined position can be chosen such that it is particularly well suited for detecting the use of the dispenser, in particular via a movement sensor or a microphone, that is to say that the data that can be captured via the at least one sensor, for example noises during use, allow reliable conclusions to be drawn about the use of the dispenser.

Preferably, a magnetizable, and in particular a metallic, holding surface is provided on the housing, while at least one permanent magnet is provided on the evaluation unit, though the reverse setup is also possible. A design in which permanent magnets are used both to create the holding surface and to make the evaluation unit magnetic is also possible.

In particular, neodymium magnets come into consideration as permanent magnets, since such magnets provide a strong magnetic force and are therefore particularly advantageous for the secure fastening of the evaluation unit on the housing of the dispenser.

The evaluation unit is preferably intended for detachable attachment to a circumferential surface of the reservoir body of the housing. The reservoir body itself preferably has a cylindrical shape in the region of the circumferential surface, in particular with a circular or an elliptical cross section.

The outer side of the reservoir body may be provided with the magnetic or magnetizable holding surface. To correspond to the curved and circular-segment-shaped or cylindrical-segment-shaped circumferential surface, the magnetic or magnetizable holding surface is preferably also of a curved form.

The evaluation unit preferably has an electronics housing which forms the contact side and within which the electronic components of the evaluation unit are arranged, that is to say in particular a processor, a memory and a battery and, depending on the type of evaluation, one or more sensors. This electronics housing does not completely surround the housing of the dispenser and in particular the reservoir body, but is preferably provided on one side of the reservoir body in the manner of a backpack, such that the dispenser, including the evaluation unit, is thereby given an asymmetrical shaping. In particular, the electronics housing preferably covers in the circumferential direction at most 50% of the circumferential surface of the reservoir body, preferably at most 40%.

The reservoir body preferably has plastic as the main material. The magnetic or magnetizable holding surface is preferably formed as a separate part, in particular as an at least partially metallic part, which is fixedly attached to the outside of a plastic wall of the reservoir body. Corresponding to the aforementioned extent of the electronics housing, this holding surface also preferably only partially surrounds the circumferential surface, in particular to an extent of at most 50%, in particular preferably to an extent of at most 40%.

The holding surface provided on the housing can be provided in various ways. It is particularly preferred if the holding surface is provided in the region of an inscribed label. It may directly form the label or be adhesively bonded to a housing surface of the housing by means of the label. The label may comprise the product designation or a product description relating to the pharmaceutical or cosmetic product contained in the media reservoir. Alternatively, the label may also contain handling instructions which describe the attachment of the evaluation unit. This may also for example take the form of a symbol which illustrates the attachment.

The holding surface may also be self-attached independently of the label to the housing by means of an adhesive connection. Designs in which the holding surface is formed by a holding sleeve or holding clamp which is attached to the housing of the dispenser in a clamping manner are also possible. Such a holding sleeve or holding clamp may be in particular completely or almost completely metallic. However, it may also be produced from plastic and have a magnetic or magnetizable insert.

According to a second aspect of the invention, an alternative design that allows convenient attachment of the evaluation unit to the housing of the dispenser is proposed. According to this alternative design, it is provided that the evaluation unit has at least two, and preferably at least three, elastically deflectable holding arms which engage around the housing and thereby have the effect of holding the evaluation unit clamped against the housing.

The holding arms preferably extend in different directions from a housing of the evaluation unit around the dispenser housing. In the relaxed state, the holding arms define a clear cross section which is smaller than the cross section of the housing, and in particular of the reservoir body, such that they are under elastic stress after attachment to the housing. To introduce the housing into the clear cross section, the holding arms are preferably bent open. This can take place directly manually on the holding arms or by means of gripping surfaces which are connected to the holding arms and, when force is applied, open the holding arms indirectly and thereby enlarge the clear cross section.

The holding arms can be flexibly deflected, so that they allow coupling to reservoir bodies of different cross-sectional shapes and in particular of different diameters.

The electronic evaluation unit is preferably intended for detachable attachment to a circumferential surface of the reservoir body of the housing. The reservoir body itself preferably has a cylindrical shape in the region of the circumferential surface, in particular with a circular or an elliptical cross section.

The evaluation unit preferably has an electronics housing, within which the electronic components of the evaluation unit are provided. From this electronics housing, the holding arms preferably extend in the circumferential direction around the circumferential surface of the reservoir body, the holding arms each spanning an angular range of at least 120°. The holding arms are fixedly attached with their respective root to said electronics housing and achieve their flexibility in that they are elastically deformable in themselves.

The individual holding arms are preferably curved. In particular, they extend in the form of a segment of a circle in order to engage around an approximately circular-cylindrical housing section, in particular a correspondingly shaped reservoir body. The holding arms preferably extend over an angular range of at least 120° such that two holding arms surrounding the housing in opposite directions engage around the housing by at least approximately 240°. The holding arms do not completely surround the housing and in particular the reservoir body. The holding arms preferably surround the housing or the reservoir body to an extent of at most 758, that is to say have a circumferential extent of at most 270°.

The diameter of the base circle of the circular segment shape in accordance with which the holding arms are shaped is preferably between 10 mm and 25 mm.

The number of holding arms is preferably at least three, with preferably two of these three holding arms extending around the dispenser housing in a first direction of extent, while the third holding arm is arranged between these two holding arms and extends around the dispenser housing in the opposite direction of extent.

Independently of the method of attachment by means of magnetism or by means of the holding arms, the evaluation unit may have a housing which is of a non-modular construction and intentionally cannot be dismantled further by the user. However, a particular embodiment of a dispenser according to the invention and of an evaluation unit according to the invention provides that the evaluation unit comprises a fastening unit and an electronics unit that represent separate structural units.

In this case, the fastening unit is designed for detachable attachment to the housing and for this purpose has the described magnetic or magnetizable design and/or the described holding arms. The electronics unit which can be separated therefrom is intended for detachable attachment to the fastening unit. The electronics unit preferably comprises the entirety of the electronic components of the evaluation unit, but at least the battery, the processor and at least one sensor.

The division of the evaluation unit into two makes it possible to use the electronics unit with different fastening units, for example those for magnetic fastening on the dispenser housing and those for fastening on the dispenser housing by holding arms described above.

The same electronics unit can accordingly be provided by configuration with different fastening units for attachment to many different dispensers.

The possibility of separating the fastening unit and the electronics unit and reassembling them in another configuration does not necessarily have to be provided for the end user. Said modularity can also be used to provide uniform electronics units with matching fastening units in accordance with requirements, and possibly fixedly connect them, already in the course of production and provision.

It is particularly preferred however if the fastening unit is designed for attachment to the electronics unit such that it can be detached without tools, and thus the possibility of changing the fastening unit is available to the end user themself. In particular, it may be provided for this purpose that the electronics unit is magnetically fastened on the fastening unit or the electronics unit is fastened on the fastening unit by means of a latching mechanism having an elastically deflectable latching lug.

The fastening unit is preferably free from electronic components. However, it may also be a carrier of sensors and/or have an electronically readable identification feature, for example an identification feature which is stored in an RFID chip of the fastening unit and can be read out by a sensor of the electronics unit. As a result, it is possible to record to which fastening unit the electronics unit is connected.

It is particularly preferred if the electronic evaluation unit has a contact side which in the attached state of the evaluation unit faces in the direction of the housing, with at least two mutually parallel contact ribs being provided on the contact side for bearing against the housing.

They are preferably precisely two or three mutually parallel contact ribs, while the individual contact ribs may also be interrupted and may consist of mutually aligned rib segments. The ribs form raised regions for bearing against the housing. The ribs preferably have a length of between 10 mm and 30 mm. The width of the ribs is preferably 5 mm or less, in particular preferably 3 mm or less. On their side facing the housing, the ribs are preferably provided with a rounded edge.

A set-back intermediate region, which separates the contact ribs from one another, is provided between the at least two contact ribs. In this intermediate region, the contact side of the evaluation unit is set back in such a way that it does not bear against the housing of the dispenser. The contact between the contact side and the housing of the dispenser is accordingly restricted to the at least two contact ribs.

An evaluation unit with such contact ribs allows reliable bearing against the housing of the dispenser, since the pressing force takes place in defined contact regions. In addition, the contact ribs allow bearing against housings of different dimensions and, in particular, reservoir bodies with differently curved outer contours.

Which radii of curvature of such outer contours are covered in each case depends on the geometry of the contact side. Preference is given to a design in which the contact ribs are at a distance from one another of at least 8 mm, with the contact ribs preferably rising up by at least 1 mm, preferably by at least 1.5 mm, above the contact surface in this intermediate region. Two contact ribs between which an intermediate region of 8 mm that is set back 1 mm is provided may be attached to housing sections with a radius of curvature greater than 10 mm without the housing section coming into contact with the contact side away from the contact ribs. With an intermediate region of 8 mm in width that is set back 1.5 mm, attachment up to a radius of curvature of approximately 6 mm is possible.

One or more of the contact ribs may be attached to an elastic extension arm, in particular to an extension arm in the form of a plastic tongue cut free on three sides, which allows resilient behavior orthogonal to the contact side. It may also be provided that one or more, but not all, of the contact ribs are deflectable in such a way, while at least one contact rib is provided as fixed and non-deflectable on the contact side of the evaluation unit. One possible setup provides for example three contact ribs, of which one or two are attached in each case to an elastic extension arm, while the third contact rib is provided as fixed in place on the contact side. A rib design with two contact ribs which are both deflectable may also be expedient.

The design with contact ribs may also have a positive effect in particular in the context of the magnetic attachment of the evaluation unit to the housing. The direction of extent of the contact ribs defines a displacement direction in which the evaluation unit can be displaced with respect to the housing in order to bring about a separation of the evaluation unit from the housing.

In addition to the dispenser together with the evaluation unit in its entirety, the invention also relates to the evaluation unit alone, which is designed for detachable attachment to a dispenser in the manner described above for detecting the use of the dispenser, in particular by using a microphone and/or a movement sensor.

In order to be attached to the housing of a dispenser in the manner described above, according to a first design the evaluation unit is designed as magnetic or magnetizable in the region of a contact side. In particular, at least one permanent magnet, in particular a neodymium magnet, may be provided on the contact side.

According to a second design, the evaluation unit has at least two, and preferably at least three, elastically deflectable holding arms which can engage around the housing, in particular a reservoir body of the housing, as intended and can thereby have the effect of holding the evaluation unit clamped against the housing.

The evaluation unit may be of modular form in the manner already described above and may have a fastening unit and an electronics unit. The fastening unit is designed for detachable attachment to the housing and is designed as magnetic or magnetizable in the region of the contact side. The electronics unit is intended for detachable attachment to the fastening unit and preferably comprises the entirety of the electrical/electronic components of the evaluation unit.

The evaluation unit may preferably comprise a number of interchangeable fastening units which are adapted to different types of dispenser housings and can be coupled to the electronics unit according to choice.

### BRIEF DESCRIPTION OF THE DRAWINGS

Further advantages and aspects of the invention will emerge from the claims and from the following description of preferred exemplary embodiments of the invention, which are explained below with reference to the figures.

FIGS. 1 and 2 show, by way of example, a liquid dispenser according to the invention having an evaluation unit according to the invention.

FIGS. 3 to 5 illustrate a first configuration of the embodiment of a magnetic connection between the evaluation unit and a housing of the liquid dispenser.

FIGS. 6 to 8 illustrate a second configuration of the embodiment of a magnetic connection between the evaluation unit and a housing of the liquid dispenser.

FIGS. 9 to 11 illustrate an embodiment of a connection between the evaluation unit and a housing of the liquid dispenser by means of holding arms provided on the evaluation unit.

FIGS. 12 to 15 show a possible modular embodiment of the evaluation unit with a fastening unit and an electronics unit and also coupling means for coupling the electronics unit to the fastening unit.

FIGS. 16 to 18 illustrate a first configuration of contact ribs on a contact side of the evaluation unit.

FIGS. 19 to 21 illustrate a first configuration of contact ribs on a contact side of the evaluation unit.

### DETAILED DESCRIPTION OF THE EXEMPLARY EMBODIMENTS

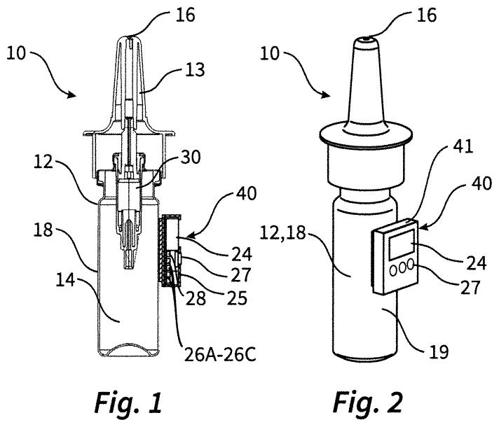

FIGS. 1 and 2 show a dispenser 10 according to the invention which is designed for discharging pharmaceutical liquids. The liquid dispenser 10 is designed by way of example as a nasal dispenser and has an applicator part with an elongate nasal applicator 13, at the distal end of which a discharge opening 16 is provided. However, the specific type of dispenser should be understood as just an example. The invention similarly also comprises other dispensers for media and in particular liquids, in particular pharmaceutical dispensers, which are designed for the oral or topical delivery of medicaments.

By depressing the applicator part with respect to a liquid reservoir 14 or the reservoir body 18 forming the liquid reservoir, it is possible to actuate a pumping device 30 which conveys liquid from the liquid reservoir 14 to the discharge opening 16.

In the case of a dispenser 10 according to the invention, it is provided that use of the dispenser 10 by the user is electronically detected. An electronic evaluation unit 40 is provided for this. In the present example, this comprises a battery 25, a display device 24, a circuit board with a processor 28, operating elements 27 and various sensors 26A to 26C.

The sensors 26A-26C may comprise in particular a movement sensor which detects the orientation of the dispenser and/or accelerations. Furthermore, the sensors 26A-26C may comprise a microphone which detects noises that occur during the handling of the dispenser 10. The captured data are evaluated either directly by means of the processor 28 or entirely or partially on an external system which is connected to the evaluation unit 40 via a radio interface.

By way of example, the processor 28 may be designed to activate the microphone in the case of movements of the dispenser 10 detected by means of the movement sensor. Noises, in particular noises transmitted through the dispenser housing 12, can be detected by means of the microphone. In particular, the different phases of a pumping process, that is to say the dispensing of liquid from a pumping chamber and the renewed aspiration of liquid from the media reservoir 14 into the pumping chamber, are accompanied by characteristic noises which are readily distinguishable from one another.

The evaluation unit 40 is attached to a circumferential surface 19 of the reservoir body 18 of the dispenser housing 12. The reservoir body 18 has a cylindrical basic shape, in the present case a circular-cylindrical basic shape.

The evaluation unit 40 does not surround the reservoir body 18, but is provided on one side in the manner of a backpack, such that it imparts to the dispenser an asymmetrical overall shape. The evaluation unit or the electronics housing 41 forming the main component span only a comparatively small part of the circumferential surface in the circumferential direction, in the present case approximately 90° or a quarter of the circumference.

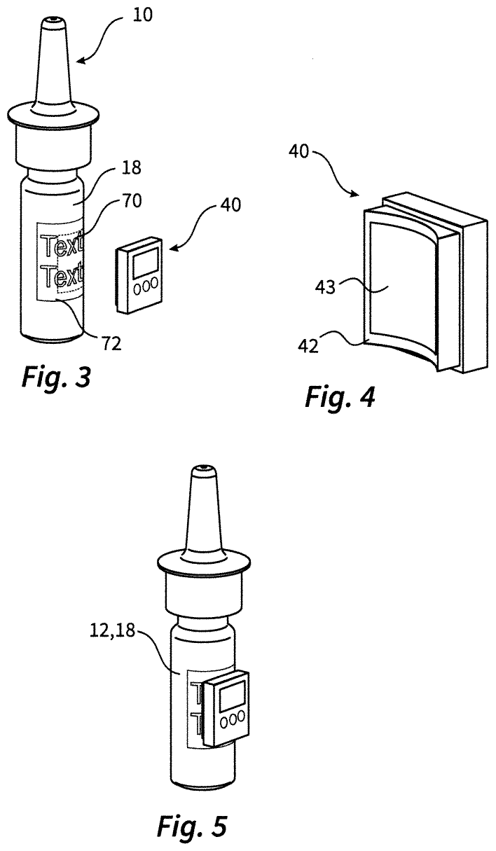

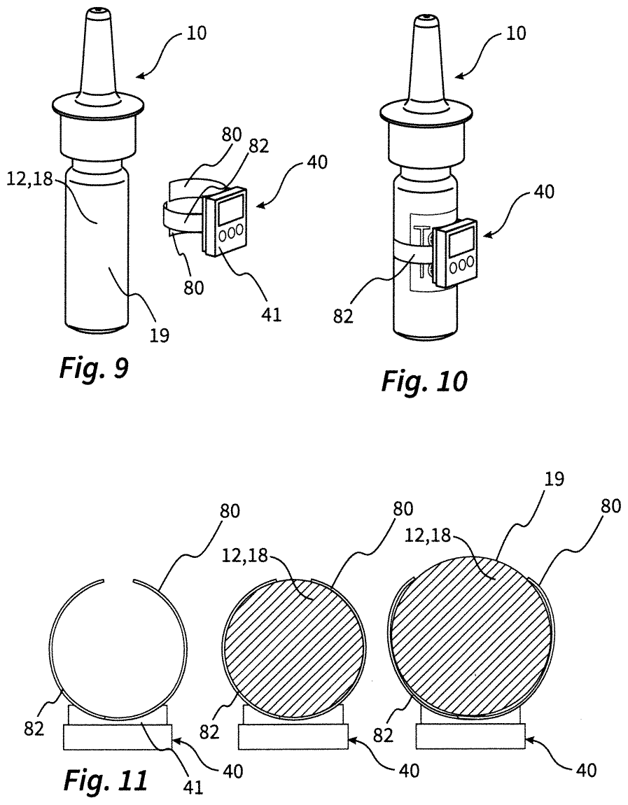

According to the invention two fastening principles, which are presented on the basis of FIGS. 3 to 8 and on the basis of FIGS. 9 to 11, are proposed for attaching the evaluation unit 40 to the reservoir body 18.

In the case of the design of FIGS. 3 to 8, it is provided that the evaluation unit 40 is magnetically fastened on the reservoir body 18.

FIG. 3 shows the reservoir body 18 of the dispenser 10, which is provided with a label 72, which may contain for example the product designation or product details or else handling instructions for attaching the evaluation unit 40. A magnetic or magnetizable holding surface 70 is provided, integrated in the label 72 or covered by the label 72. In particular, it may be a rectangular metallic sheet-metal section.

As shown in FIG. 4, a permanent magnet 43 is correspondingly provided on a contact side 42 of the evaluation unit 40. The shape and size of the holding surface 70 and of the permanent magnet 43 are preferably made to match one another in such a way that the evaluation unit 40 is held in a defined position on the reservoir body 18. The state connected in this way is shown in FIG. 5.

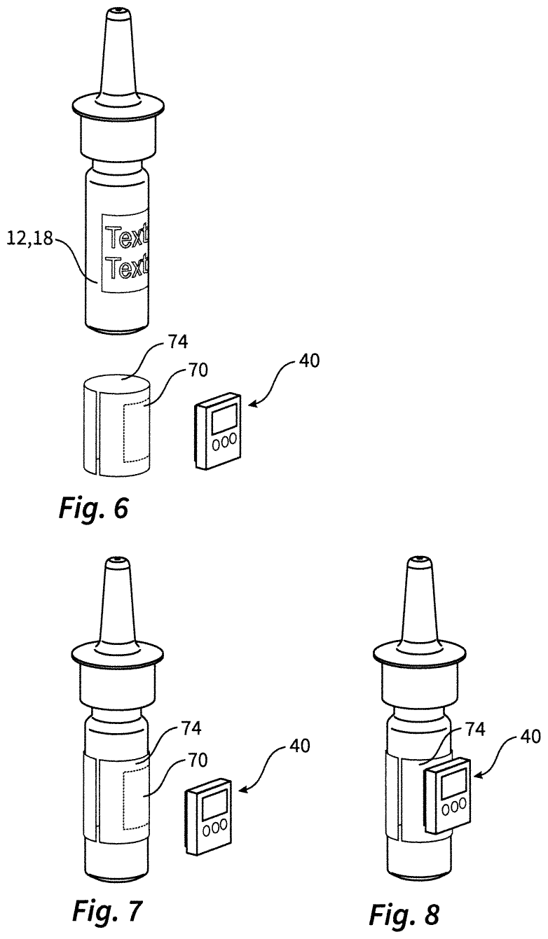

FIG. 6 shows a design in which a holding sleeve 74 is provided, designed as magnetizable completely or at least in the region of a holding surface 70. This holding sleeve 74 is designed as a slotted sleeve and can be pushed onto the reservoir body 18 in the manner illustrated in FIG. 7, with the inner diameter of the holding sleeve 74 being adapted to the outer diameter of the reservoir body 18 in such a way that, after being pushed onto the reservoir body 18, the holding sleeve 74 is elastically widened and as a result is held on the reservoir body 18 in a clamping manner.

The holding surface 70 as part of the holding sleeve allows the magnetic attachment of the evaluation unit 40 in the manner illustrated in FIG. 8.

FIGS. 9 to 11 show another form of fastening an evaluation unit 40 on the reservoir body 18. As can be seen on the basis of FIG. 9, in the case of this design it is provided that altogether three holding arms 80, 82 are provided on the housing of the evaluation unit, each having a curved shape and jointly surrounding a region for receiving the reservoir body 18. The holding arms 80, 82 are fixedly attached with their root to the electronics housing 41.

The upper and the lower holding arm 80 extend counter-clockwise from the housing of the evaluation unit 40. The holding arm 82 lying in between extends clockwise around the housing of the evaluation unit 40.

In the non-attached state of FIG. 9, the holding arms 80, 82 are not elastically deflected. If however the reservoir body 18 is pushed into the region surrounded by the holding arms, the holding arms are thereby pressed slightly apart such that they bear against the outer side of the reservoir body in a clamping manner and thereby create a secure coupling. The assembled state is shown in FIG. 10.

FIG. 11 illustrates how the evaluation unit can be attached to housings 12 of different diameters. The representation on the left of FIG. 11 shows the evaluation unit 40 in the unused state. The representation in the middle of FIG. 11 shows the evaluation unit 40 after coupling to a housing 12, the outer diameter of which is only slightly larger than the rest state of the holding arms 80, 82. As a result, the holding arms 80, 82 are elastically deflected outward and therefore clamp the reservoir body 18 in. The representation on the right in FIG. 11 shows the use of the evaluation unit 40 with a significantly larger housing 12. The holding arms 80, 82 are widened significantly further here. As a result, they do not bear against the outer side of the housing 12 over their entire length. Since however the deflection and the accompanying restoring force are considerably greater, a secure hold is nevertheless also achieved on this housing 12.

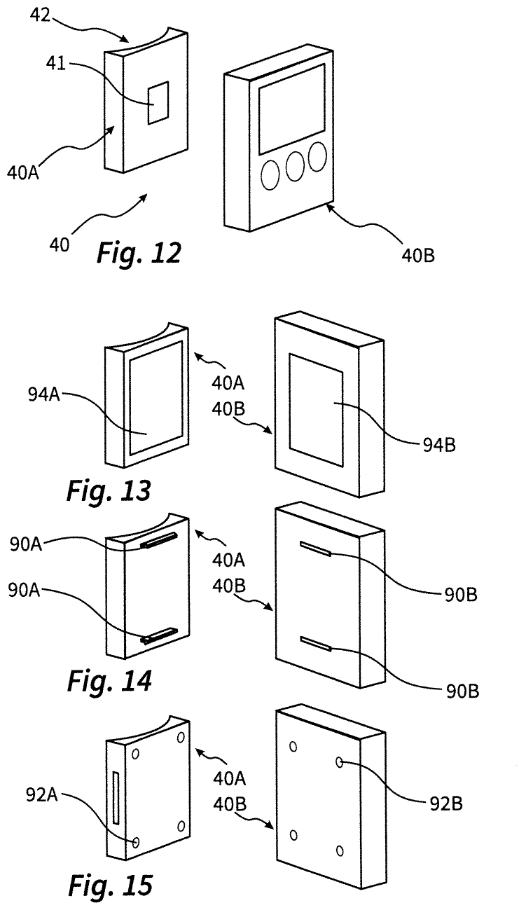

FIGS. 12 to 15 illustrate the possibility of designing the evaluation unit 40 in a modular manner. In a manner not illustrated in any more detail, the contact side 12 of the evaluation unit 40 may be designed as magnetic or magnetizable in accordance with FIGS. 3 to 8 or may be provided with holding arms in accordance with FIGS. 9 to 11.

As illustrated on the basis of FIG. 12, in such cases the evaluation unit 40 is subdivided into a fastening unit 40A, on which the contact side 42 is provided, and an electronics unit 40B, which has the predominant number of electrical/electronic components, preferably the entirety of the electronic components. It may however be provided that the fastening unit 40A has an electronic identification means such as an RFID chip 41 in order that the electronics unit 40B can detect whether it is connected to a fastening unit 40A or to which fastening unit 40A it is connected.

The subunits 40A, 40B can preferably be coupled to one another without tools. In the case of the design of FIG. 13, the two subunits are connected to magnetic or magnetizable coupling surfaces 94A, 94B. In the case of such a design, it is possible to provide a common magnet on the fastening unit 40A, which serves for coupling to the housing 12 and for coupling to the electronics unit 40B.

In the case of the design of FIG. 13, latching lugs 90A which latch into latching openings 90B on the electronics unit 40B are provided on the fastening unit 40A. Preferably, as a difference from the design of FIG. 20, the latching lugs 90A can be deflected manually in order to be able to separate the connection again. It may however also be provided that the connection of the electronics unit 40B to the fastening unit 40A is intentionally undetachable such that a corresponding coupling is possible only once, in particular in the case of fixing onto a specific fastening unit 40A that already takes place in the course of production.

In the case of the design of FIG. 14, screw holes 92A, 92B are provided on the electronics unit 40B and the fastening unit 40A, through which the two subunits can be screwed to one another. This type of connection is usually not intended to be disconnected by the end user, but serves for the purpose of being able to connect a uniform electronics unit 40B permanently to a fastening unit 40A in the course of production.

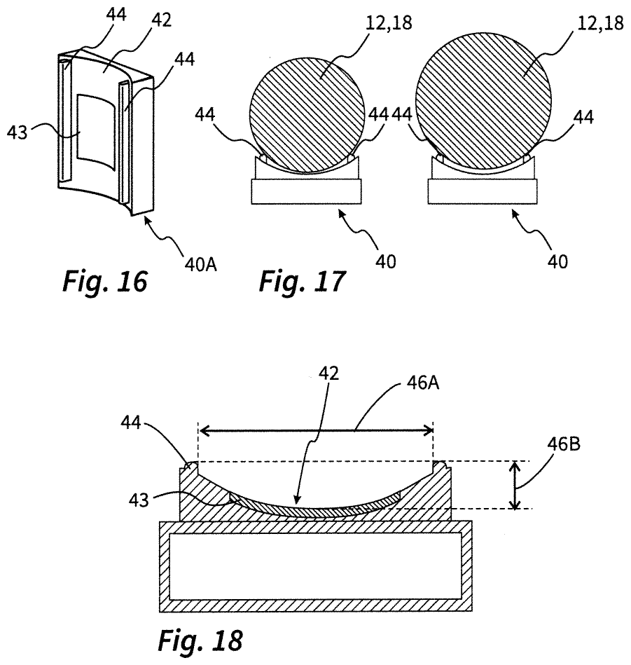

FIGS. 16 to 21 illustrate that it may be advantageous if the evaluation unit 40 or its fastening unit 40A is provided with contact ribs 44. These are intended for bearing against the housing 12 or the holding sleeve 74.

FIGS. 16 to 21 show a first configuration of contact ribs 44. FIG. 16 shows the rear side of the evaluation unit 40. This rear side forms a contact side 42, with two parallel contact ribs 44 being provided as fixed in place in relation to one another on the contact side 42. A set-back intermediate region is provided between the two contact ribs 44, with a permanent magnet 43 being fastened here. As can be seen on the basis of FIG. 17, the contact ribs 44 make it possible to bear against dispenser housings 12 of different radii of curvature. The same evaluation unit 40 or the same fastening unit 40A of the evaluation unit 40 can therefore be used equally well for different dispensers 10.

FIG. 18 once again illustrates the geometry of the contact side 42. It can be seen that it has a basically curved concave shape and that the two contact ribs 44 rise up above this curved concave shape. The distance 46A corresponds to the width of the intermediate region. This distance is smaller than the diameter of the reservoir body 18 in order to fulfill the rib function. It is preferably at most 80% of the diameter, based on the horizontal diameter in the alignment of FIG. 17.

The distance 46B indicates the extent to which the intermediate region is set back at most with respect to the contact ribs 44. Since the permanent magnet 43 is also arranged in the intermediate region, it is at some distance from the outer side of the dispenser housing 12 owing to the contact ribs 44. However, if sufficiently strong magnets, in particular neodymium magnets, are chosen, this distance does not preclude there being a sufficient holding force.

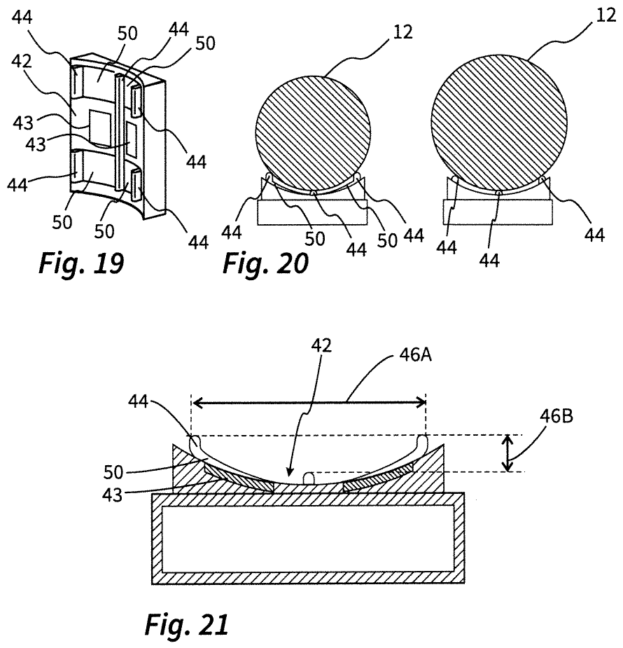

FIGS. 19 to 21 show a second configuration of contact ribs 44. As can be seen on the basis of FIG. 19, the contact side 42 is provided here with altogether three contact ribs 44. A central contact rib 44 is fixedly connected to the housing of the evaluation unit 40 and cannot be deflected to a relevant extent with respect thereto. By contrast, two lateral contact ribs 44 are provided from extension arms 50, with both lateral contact ribs 44 each consisting of two mutually aligned rib segments, which for their part are in each case provided on an extension arm 50. The two outer contact ribs can therefore be deflected in a resilient manner.

The extension arms 50 allow the outer contact ribs 44 to be displaced relative to one another against the spring force of the extension arms 50. As a result, although there are altogether three contact ribs, it can be achieved that all of the contact ribs bear in each case against the dispenser housing 12 when the evaluation unit 40 is used with different dispenser housings 12 or reservoir bodies 18. In the case of dispenser housings 12/reservoir bodies 18 with larger diameters, the extension arms 50 are elastically deflected.

1. A dispenser for discharging pharmaceutical or cosmetic media, comprising:
a housing;
a media reservoir;
a discharge opening, wherein medium from the media reservoir is deliverable through the discharge opening into a surrounding atmosphere;
an electronic evaluation unit for detecting use of the dispenser,
the electronic evaluation unit being configured for detachable attachment to the housing of the dispenser, the electronic evaluation unit comprising a contact side; and
a magnetic or magnetizable holding surface provided on an outer side of the housing;
wherein the contact side of the electronic evaluation unit is magnetic or magnetizable such that the electronic evaluation unit is magnetically fastenable on the housing.

2. The dispenser as claimed in claim 1, wherein the housing includes a reservoir body having an outer circumferential surface including a magnetic or magnetizable holding surface,
the electronic evaluation unit being configured for detachable attachment to the outer circumferential surface of the reservoir body.

3. The dispenser as claimed in claim 2, wherein
the outer circumferential surface of the reservoir body and the magnetic or magnetizable holding surface together have a curved shaping.

4. The dispenser as claimed in claim 2, wherein the evaluation unit includes electronic components and
an electronics housing, the electronic components being arranged in the electronics housing, and
the electronics housing covers, in a circumferential direction, at most 50% of a total circumferential surface of the reservoir body.

5. The dispenser as claimed in claim 2, wherein the
reservoir body comprises primarily plastic and has a plastic wall with an outside, and
the magnetic or magnetizable holding surface is fixedly attached to the outside of the plastic wall of the reservoir body.

6. The dispenser as claimed in claim 2, wherein
the magnetic or magnetizable holding surface is covered by an inscribed label or is integrated into an inscribed label.

7. The dispenser as claimed in claim 6, wherein
the magnetic or magnetizable holding surface is formed by a holding sleeve or a holding clamp fastened on the housing in a clamping manner.

8. The dispenser as claimed in claim 2, wherein
the magnetic or magnetizable holding surface is adhesively fastened on the housing.

9. A dispenser for discharging pharmaceutical or cosmetic media, comprising:
a housing;
a media reservoir;
a discharge opening, wherein medium from the media reservoir is deliverable into a surrounding atmosphere through the discharge opening; and
an electronic evaluation unit for detecting use of the dispenser,
the electronic evaluation unit being designed for detachable attachment to the housing of the dispenser,
the evaluation unit having at least two elastically deflectable holding arms engaging around the housing and clamping the evaluation unit against the housing.

10. The dispenser as claimed in claim 9, wherein the housing comprises a reservoir body having a circumferential surface,
the electronic evaluation unit is configured for detachable attachment to the circumferential surface of the reservoir body of the housing,
the evaluation unit has electronic components and an electronics housing and the electronic components of the evaluation unit are arranged in the electronics housing, and
the holding arms extend in a circumferential direction from the electronics housing around the circumferential surface of the reservoir body, with the holding arms each spanning an angular range of at least 120°.

11. The dispenser as claimed in claim 9, wherein
the holding arms each span an angular range of at most 270°.

12. The dispenser as claimed in claim 10, wherein
the electronics housing covers in the circumferential direction at most 50% of the circumferential surface of the reservoir body, and
the holding arms are fixedly attached to the electronics housing and extend from the electronics housing in different directions.

13. The dispenser as claimed in claim 5, further comprising
at least three holding arms wherein two of the three holding arms extend in a first direction of extent and third of the three holding arms is arranged between the two holding arms and extends in a second direction of extent opposite the first direction of extent.

14. The dispenser as claimed in claim 13, wherein
the holding arms extend in the form of a segment of a circle.

15. The dispenser as claimed in claim 1, wherein
the evaluation unit comprises a fastening unit and an electronics unit,
the fastening unit is configured for detachable attachment to the housing and
the electronics unit is configured for detachable attachment to the fastening unit.

16. The dispenser as claimed in claim 15, further including at least one of the following:
the fastening unit is configured for attachment to the housing such that the fastening unit is detachable from the housing without tools; and/or
the electronics unit is magnetically fastened on the attachment unit; and/or
the electronics unit is fastened on the fastening unit by a latching mechanism having an elastically deflectable latching lug; and/or
the fastening unit includes an identification feature stored in an RFID chip of the fastening unit, the identification feature being readable by a sensor of the electronics unit.

17. A dispenser for discharging pharmaceutical or cosmetic media, comprising:
a housing; and
an electronic evaluation unit for detecting use of the dispenser, the electronic evaluation unit being configured for detachable attachment to the housing,
the electronic evaluation unit having a contact side, the contact side, in an attached state of the evaluation unit, facing in a direction of the housing, and
the evaluation unit has on the contact side two mutually parallel contact ribs for bearing against the housing.

18. The dispenser as claimed in claim 17, including at least one of the following:
the ribs are located at a distance from one another of at least 8 mm; and/or
the ribs project outwardly by a distance of at least 1.5 mm with respect to an intermediate region lying between the ribs; and/or
at least one of the ribs is attached to an elastic extension arm; and/or
at least one of the ribs is formed by a number of mutually aligned rib segments, each of the rib segments being disposed on an elastic extension arm.

19. The dispenser as claimed in claim 1, including at least one of the following:
the evaluation unit comprises at least one sensor comprising a movement sensor and/or a microphone; and/or
the evaluation unit comprises an output device comprising at least one of: an LED; a display; a vibrator; and/or a loudspeaker; and/or
the evaluation unit comprises a radio module comprising a Bluetooth and/or WIFI radio module; and/or
the evaluation unit comprises an energy source comprising a battery.

20. The dispenser as claimed in claim 1, wherein:
the dispenser is configured as a liquid dispenser with the media reservoir being configured as a liquid reservoir;
the dispenser further including at least one of the following:
the dispenser is configured as a squeeze-bottle dispenser and has an elastically deformable reservoir body, wherein manual compression of the reservoir body discharges liquid; and/or
the dispenser is configured as a pumping dispenser having a manually actuable pumping device conveying liquid from the liquid reservoir to the discharge opening; and/or
the dispenser is configured as a spray dispenser; and/or
the dispenser is configured as an inhaler having a container depressible for delivering a dose of liquid; and/or
the dispenser is configured as a drop dispenser having a drop-formation surface surrounding the discharge opening; and/or
the dispenser is configured as a nasal dispenser having an elongate nasal applicator with a distal end, the discharge opening being disposed at the distal end.

21. An electronic evaluation unit for a dispenser having a housing, the evaluation unit being configured for
detachable attachment to the dispenser, the evaluation unit comprising:
at least one sensor; and
one of more of the following:
a contact side configured to be magnetic or magnetizable such that the evaluation unit is magnetically fastenable on the dispenser housing; and/or
at least two elastically deflectable holding arms engageable, in use, around the dispenser housing to clamp the evaluation unit against the dispenser housing.

22. The evaluation unit as claimed in claim 21, further comprising
a fastening unit and an electronics unit, the fastening unit being configured for detachable attachment to the dispenser housing and with the electronics unit being configured for detachable attachment to the fastening unit.

23. The dispenser as claimed in claim 1, wherein the housing includes a reservoir body, and the holding surface is disposed on the reservoir body of the housing.

24. The dispenser as claimed in claim 6, including at least one of the following:
the inscribed label comprises a product designation and/or a product description; and/or
the inscribed label comprises handling instructions relating to attachment of the evaluation unit.

25. The dispenser as claimed in claim 9, wherein the housing includes a reservoir body, and the deflectable holding arms engage around the reservoir body.

26. The dispenser as claimed in claim 17, further comprising a media reservoir having a reservoir body, and the contact ribs bear against the reservoir body.

27. The evaluation as claimed in claim 22, wherein the evaluation unit comprises a plurality of fastening units configured for attachment to the electronics unit.

28. A dispenser for discharging pharmaceutical or cosmetic media, comprising:
a housing;
a media reservoir;
a discharge opening, wherein medium from the media reservoir is deliverable through the discharge opening into a surrounding atmosphere;
an electronic evaluation unit for detecting use of the dispenser and configured for detachable attachment to the housing of the dispenser, the electronic evaluation unit comprising a contact side, the contact side being magnetic or magnetizable; and
a magnetic or magnetizable holding surface disposed on an outer side of the housing, the magnetic or magnetizable holding surface being covered by, or integrated into, an inscribed label, the inscribed label comprising one of: a product designation or a product description; or instructions for attachment of the electronic evaluation unit to the dispenser, the contact side of the electronic evaluation unit being magnetically fastenable to the magnetic or magnetizable holding surface of the housing.
